(19) United States
(12) Patent Application Publication
(10) Pub. No.: US 2012/0017916 A1
(43) Pub. Date: Jan. 26, 2012

(54) Dilator With Integrated Guiding Catheter

(76) Inventors: James F. Schumacher, Cumming, GA (US); Sam C. Chan, Atlanta, GA (US); Nathan C. Griffith, Roswell, GA (US); F. Anthony Headley, JR., Atlanta, GA (US)

(21) Appl. No.: 12/843,425

(22) Filed: Jul. 26, 2010

(51) Int. Cl. A61M 16/00

(52) U.S. Cl. 128/207.29

(57) ABSTRACT

There is provided a device for performing a tracheotomy. The tracheostomy dilator has a body, tip and guiding catheter which are non-detachably attached to each other. The dilator has a cannula sized to accommodate a guide wire. After the trachea has been dilated, the entire device may be removed, leaving only the guide wire in the stoma, and a tracheostomy tube may be inserted over the guide wire and into the trachea. This substantially reduces the number of components and steps required for a successful tracheostomy procedure.

Ventilators or respirators are used for mechanical ventilation of the lungs of a patient in a medical setting. The ventilator unit is connected to a hose set; the ventilation tubing or tubing circuit, delivering the ventilation gas to the patient. At the patient end, the ventilation tubing is typically connected to a tracheal ventilation catheter or tube, granting direct and secure access to the lower airways of a patient. Tracheal catheters are equipped with an inflated sealing balloon element, or “cuff”, creating a seal between the tracheal wall and tracheal ventilation tube shaft, permitting positive pressure ventilation of the lungs.

One type of tracheal catheter, an endotracheal tube (ET tube), inserted through the mouth, is generally used for a number of days before a decision is made to switch a patient to a tracheostomy tube, inserted directly into the trachea through a stoma in the tracheal wall. Endotracheal tubes have been linked in some studies to an increased rate of ventilator acquired pneumonia (VAP) and so tracheostomy operations are becoming increasingly common and are being performed earlier in the patient's hospital stay in order to reduce the occurrence of VAP.

A tracheostomy procedure involves making a small horizontal incision in the skin of the neck to grant access to the trachea. Because of the uniquely flexible and elastic nature of the trachea, it has been found that healing is much faster if only a small hole is made in the tracheal wall and the hole dilated, rather than cutting the tracheal wall. After the skin incision, a hemostat or other implement may be used to separate the subcutaneous tissues to gain access to the trachea, and digital palpation is used to locate the tracheal rings. A bronchoscope is usually inserted into the ET tube and the tube withdrawn from the trachea until the light of the bronchoscope transdermally illuminates the site of the incision. A sheathed needle is used to puncture the tracheal wall, usually between the second and third tracheal rings. The needle is removed with the sheath remaining, a flexible guide wire (also called a J-wire) is inserted in the place of the needle and the sheath is removed. The bronchoscope is used for viewing the procedure from within the trachea in order to avoid damage to the tracheal wall. A small (e.g. 14 French) introducer dilator is introduced over the guide wire to perform an initial dilation of the tracheal wall, and then removed. A smaller (e.g. 8 French) guiding catheter is then introduced over the guide wire. (Note, French is a measure of circumference based on the theory that non-round tubes of the same circumference will fit into the same incision. One French is approximately 0.33 mm or 0.013 inch).

After the guiding catheter is introduced, a first dilator such as the Cook Medical Inc. Blue Rhino® dilator (see also U.S. Pat. No. 6,637,435), is placed over the guide wire and the guiding catheter and first dilator are advanced into the trachea through the tracheal wall as a unit to perform the dilation. Cook Medical recommends a slight over-dilation of the tracheal wall in order to make the placement of the tracheostomy tube easier. After dilation, the first dilator is removed and the tracheostomy tube (with cannula removed) is introduced over the guide catheter using a second, loading dilator that fits just inside the trachostomy tube and protrudes about 2 cm beyond the distal end of the tracheostomy tube. The guide catheter, second dilator and tracheostomy tube are advanced into the trachea through the tracheal wall as a unit. Once the tracheostomy tube is at the proper depth, the second dilator, guide catheter and guide wire are removed through the tracheostomy tube, the inner cannula inserted into the tracheostomy tube and the tube connected to the ventilator.

As can be understood from the above description, the current state of the art for tracheostomy involves numerous steps and the insertion and removal of a number of components before the successful completion of the procedure. For most of this time, the patient is disconnected from the ventilator and is therefore, not breathing. In addition, the large number of parts used in current tracheostomy kits increases the likelihood that an item may be accidentally rendered unsterile and be unable to be used. In such cases, the patient must be re-intubated with an ET tube. Even if the procedure proceeds uneventfully, however, the amount of time the patient is not breathing is significant; on the order of 7 minutes or more. This is clearly a significant event, especially for a patient who is, most likely, not in optimal physical condition.

There remains a need for a device that can more quickly and safely allow for the successful placement of a tracheostomy tube.

### SUMMARY OF THE INVENTION

There is provided a device for performing a tracheostomy. The described tracheostomy dilator has a body portion, a tip portion and a guiding catheter portion that are an integral part of the dilator. After the trachea has been dilated, the entire device may be removed from the patient, leaving only the guide wire in the stoma. A tracheal tube may be inserted over the guide wire and into the trachea.

### BRIEF DESCRIPTION OF THE DRAWINGS

FIG. 1 is a drawing of the prior art Blue Rhino® dilator.

FIG. 2 is a drawing of the device.

FIG. 3 is a drawing of the device being moved into the trachea through the tracheal wall.

FIG. 4 is a drawing of the device after being inserted into the trachea through the tracheal wall to the point where the “stop” mark or insertion depth gauge, meets the incision.

FIG. 5 is a drawing of the device being immersed in water to activate the lubricious coating, and also showing the alignment line and the surface formation or “grip dimples” that allow for a better hand-hold on the device.

### DETAILED DESCRIPTION OF THE INVENTION

Tracheostomy is a lifesaving procedure to allow a patient to be ventilated directly through the trachea. Tracheostomy is also believed by many to prevent or retard the onset of ventilator acquired pneumonia (VAP). This lifesaving procedure, unfortunately, is relatively time consuming and current technology requires a large number of steps and pieces of equipment that must remain sterile and functioning properly in order to arrive at a successful conclusion.

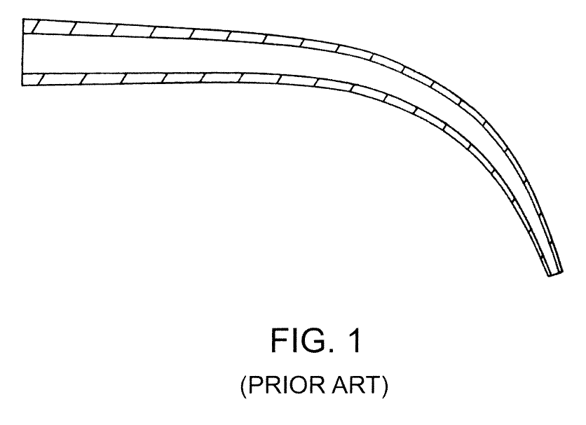

Dilators are instruments or substances for enlarging a canal, cavity, blood vessel or opening, according to the American Heritage Stedman's Medical dictionary 2001. FIG. 1 is a drawing of the prior art dilator from Cook Medical Inc. known as the Blue Rhino® dilator (see also U.S. Pat. No. 6,637,435). This patent describes a one piece dilator having a generally linear shaft and a short distal tip portion with a curved tapered portion in between.

The tracheostomy procedure may be greatly improved using the device described in the Summary above (the device). The device replaces a number of pieces used in the current state of the art procedure described in the introduction. The device replaces both the first and second dilators and the guiding catheter and so provides fewer steps in the procedure, saving time and reducing risk to the patient.

By including a guiding catheter as part of the device, the device provides a visual guide for the physician during insertion of the device. Previously, physicians had to be careful to watch both the guide wire and guiding catheter during dilation. The problem with the guiding catheter is that it could move proximally into the body of the dilator, leaving no guide for the dilator. Conversely, the guide wire could move distally into the trachea of the patient during dilation, pushed by the dilator. Incorporating the guiding catheter into the dilator removes at least one of these concerns, allowing the physician to concentrate his attention on the dilation procedure.

The device also has a number of other novel features to help ensure the consistency and ease of the procedure for the physician.

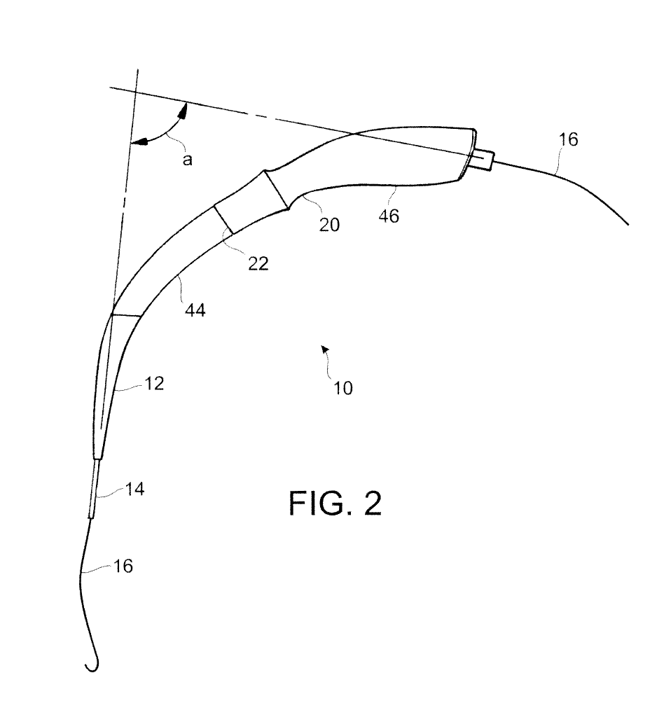

Turning to the Figures, one embodiment of the device 10 has a body 20 portion, a distal tip 12 portion and a guiding catheter 14 portion (FIG. 2). The device 10 is a single part or piece wherein the tip 12 is non-detachably attached to the body 20. The tip 12 is also non-detachably attached to a guiding catheter 14 and is desirably tapered. The body 20 has a marking line 22 or alternatively a ridge where the diameter is approximately 42 French which serves as a depth marking or insertion stopping point for the dilation procedure. The body 20 has a distal portion 44 and a handle portion 46. Guide wires are generally between about 0.2 and 0.5 mm in diameter and the guiding catheter should be just slightly larger than the guide wire, or about 8 to 11 French.

It should be noted that, although the dilator is described as having non-detachable body, tip and guiding catheter portions, implying separate pieces, one single piece dilator could be made, having approximately the same dimensions as the three portions. Both embodiments are intended to be encompassed by the specification and accompanying claims.

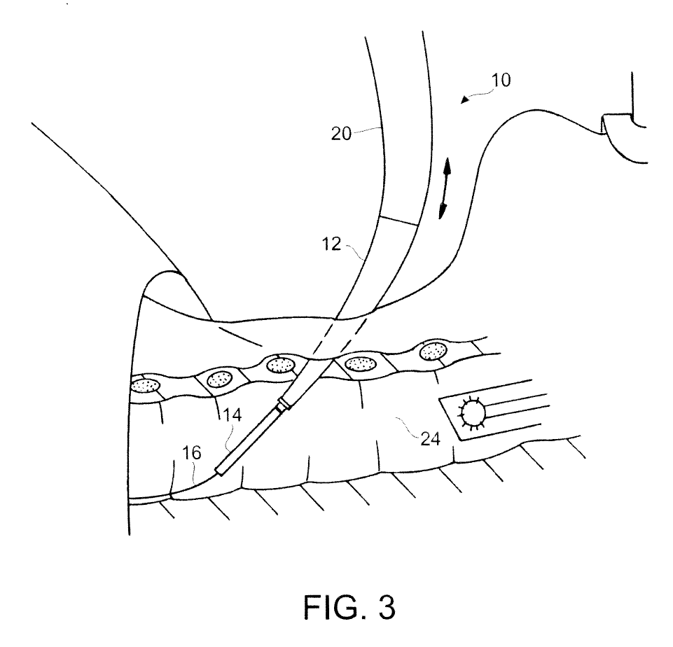

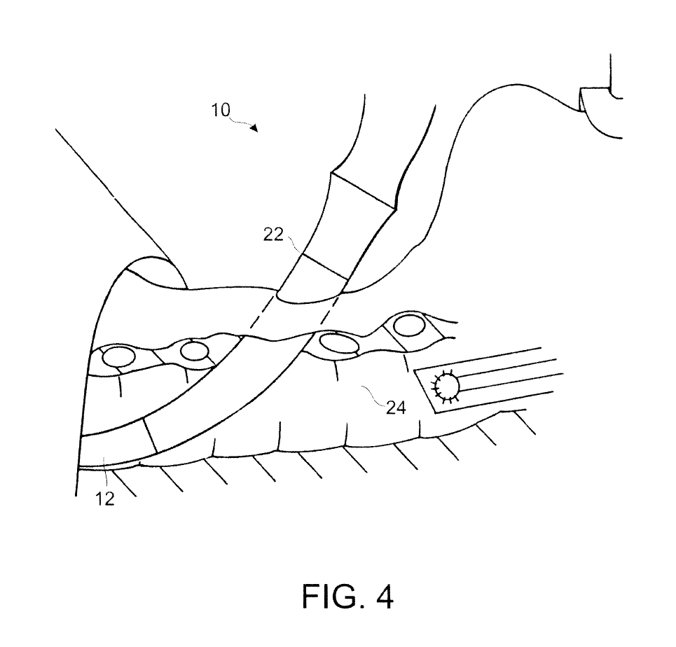

As described above, once the guide wire 16 is inserted into the trachea 24 through the incision 32 and tracheal wall 34, the device 10 is introduced over the guide wire 16. The device 10 is then moved into the trachea 24 through the tracheal wall 34 until the marking line 22 of the device 10, which serves as a “stop” mark or depth gauge, meets the incision 32 (FIGS. 3 and 4 sequentially). The actual procedure of dilation of the tracheal wall usually involves the repeated incremental insertion and removal of the device 10. This procedure may be made easier for the medical provider and less traumatic for the patient by the application of a lubricious coating to the device 10. The coating can reduce friction and drag on the guide wire 16 and also reduce trauma to the area of the incision 32 and the tracheal wall 34. This coating is described in more detail below.

Once the trachea 24 is satisfactorily dilated, the device 10 removed from the trachea 24, leaving only the guide wire 14 in the stoma. After removal of the device 10, a tracheostomy tube (not shown) may be passed axially over guide wire 14 and then into the trachea 24. Once the tracheostomy tube is in place, the sealing cuff on the distal portion of the tube is inflated and the tube is connected to a ventilator and placed in service in the conventional manner.

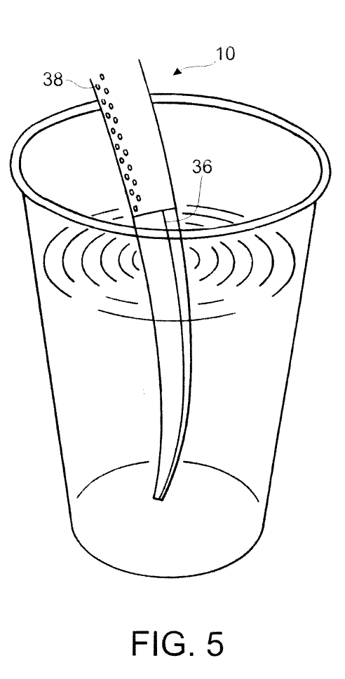

In addition to the above features, the device may have a number of other features to aid the physician in placement of the dilator, some of which are illustrated in FIG. 5. One optional feature is a guiding line 36 running length-wise on the uppermost surface of the dilator that allows one to align the guide wire, which also has a line, so that it is facing in the proper direction with the J-loop facing downward. A second optional feature is the use of surface formations 38 to enhance the grip. Such surface formations may be notches, chevrons, “dimples” or other shapes on the dilator body placed in the area where the dilator body would be gripped during a tracheostomy. While the surface formations may be raised from the surface of the dilator body, it is desirable that the surface formations be recessed into the body so as to reduce trauma to tissue. Another optional feature is a ridge 42 located proximal to the marking line 22 as shown in FIGS. 3 and 4. The dilator body 20 continues to enlarge between the marking line 22 and the ridge 42 so that the tracheal wall may be “over dilated” as preferred by some medical providers. The proximal side of the ridge 42 also serves as a convenient holding point for the user's thumb and fingers during the procedure so that the body 20 may be held like a pencil. The marking line 22 at 42 French on the dilator body may instead be an additional ridge or other marking and alternate or additional markings may be placed on the dilator body at, for example, 32, 38 or still larger French diameters.

Another optional trauma reducing feature is a lubricious coating that may be added to the tip and dilator body up to the stop ridge on the exterior and/or interior. The coating may be activated by exposure to water (FIG. 5) before the device 10 is slipped over the guiding catheter 14. The coating may be for example, a poly(N-vinyl) lactam such as those available from Hydromer Inc., 35 Industrial Parkway, Branchburg, N.J. and as described in U.S. Pat. Nos. 5,156,601, 5,258,421, 5,420,197 and 6,054,504. The dilator may be dipped in water just before the guide wire is inserted and may be coated on the inside and/or outside. An inside coating allows the guide wire to slip through the interior of the dilator quite easily and the exterior coating avoids trauma to the skin or trachea.

The device should be made from a pliable, flexible material so that it is firm enough to enter the trachea and dilate the tracheal wall, yet not so rigid and firm that it will not flex or “give” when it meets an obstruction. The flexibility of the parts of the device may vary, furthermore, with the guiding catheter 14 portion being the most flexible, the proximal end of the body being the least flexible and the flexibility of the body varying between the two. Polymers that may be suitable for use in making the device include polyolefins, polycaprolactones, polyurethanes and others. Polyurethane has been found to be particularly useful in producing the device. The device must be biocompatible, free of di(2-ethylhexyl)phthalate (DEHP) and preferably free of animal derived products.

In contrast to the prior art dilator (FIG. 1), the dilator body described herein should be substantially curved (FIG. 2) so that the proximal end will have less likelihood of contacting the chin of the patient during the procedure. By “substantially curved” it is meant that while small sections of the device may be or appear straight, the overall shape of the device is clearly curved. As can be seen in FIG. 2, for example, when the device 10 is inserted over the guide wire 16, the guide wire 16 exits the proximal end of the body 20 at an angle “a” from the distal end of the guide wire 16 which, in this instance, is 84 degrees. The degree of curvature may vary, however, and still be considered within the scope of the invention. This angle “a” may be between 110 and 60 degrees, more particularly between 100 and 70 degrees, still more particularly between 90 and 80 degrees.

Exemplary sizes for the various components of the dilator and loading catheter are as follows;

The dilator body 20 portion, for example, should have a total length of less than 28 cm. The dilator tip 12 portion may be between about 25 and 80 mm in length, particularly about 35 mm long, tapering from 3 to 6 mm at the distal end to about 5 to 16 mm, particularly 4 mm at the distal end to 8 mm. The guiding catheter 14 portion may be between 1 and 5 cm in length.

This application is one of two commonly assigned patent applications which are being filed on the same day. The group includes application Ser. No. ______ (attorney docket no. 64676201 US01) in the name of James F. Schumacher and is entitled “Tracheostomy Tube Loading Catheter”.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A one piece tracheostomy dilator comprising a guiding catheter portion, a tip portion and a body portion, said dilator having a cannula adapted to receive a guide wire.

2. The dilator of claim 1 wherein an angle formed by lines from a distal end of said guide wire and a proximal end of said guide wire passing through said dilator is between 110 and 60 degrees.

3. The dilator of claim 1 wherein an angle formed by lines from a distal end of said guide wire and a proximal end of said guide wire passing through said dilator is between 100 and 70 degrees.

4. The dilator of claim 1 wherein an angle formed by lines from a distal end of said guide wire and a proximal end of said guide wire passing through said dilator is between 90 and 80 degrees.

5. The dilator of claim 1 having surface formations on the proximal end of said body to enhance the grip of the user.

6. The dilator of claim 1 having a water activated lubricious coating on a surface.

7. The dilator of claim 1 which changes from relatively very flexible at a distal end to relatively less flexible at a proximal end.

8. The dilator of claim 1 comprising a guiding line running length-wise on an uppermost surface of said dilator.

9. The dilator of claim 1 wherein said tip is between 25 and 80 mm in length and said body is between 12 and 25 cm in length.

10. The dilator of claim 1 wherein said body has a guiding line.
